SEQUENCE LISTING (7 sequences)

SEQ ID NO: 1 - DNA, 11 bp, synthetic construct
  misc_feature 1..11: note = Description of Artificial Sequence: Synthetic oligonucleotide
  source 1..11: mol_type = other DNA
aaaaaattttt

SEQ ID NO: 2 - DNA, 11 bp, synthetic construct
  misc_feature 1..11: note = Description of Artificial Sequence: Synthetic oligonucleotide
  source 1..11: mol_type = other DNA
aaaaatttttt

SEQ ID NO: 3 - DNA, 12 bp, synthetic construct
  misc_feature 1..12: note = Description of Artificial Sequence: Synthetic oligonucleotide
  source 1..12: mol_type = other DNA
aaaaaacttttt

SEQ ID NO: 4 - DNA, 10 bp, synthetic construct
  misc_feature 1..10: note = Description of Artificial Sequence: Synthetic oligonucleotide
  source 1..10: mol_type = other DNA
aaaaactttt

SEQ ID NO: 5 - DNA, 10 bp, synthetic construct
  misc_feature 1..10: note = Description of Artificial Sequence: Synthetic oligonucleotide
  source 1..10: mol_type = other DNA
tcagtcccga

SEQ ID NO: 6 - DNA, 11 bp, synthetic construct
  misc_feature 1..11: note = Description of Artificial Sequence: Synthetic oligonucleotide
  source 1..11: mol_type = other DNA
tcagtccccga

SEQ ID NO: 7 - DNA, 12 bp, synthetic construct
  misc_feature 1..12: note = Description of Artificial Sequence: Synthetic oligonucleotide
  source 1..12: mol_type = other DNA
tcagtcccccga

(19) United States
(12) Patent Application Publication
(10) Pub. No.: US 2025/0061970 A1
(43) Pub. Date: Feb. 20, 2025

(54) SYSTEMS AND METHODS FOR DETECTING HOMOPOLYMER INSERTIONS/DELETIONS

(71) Applicant: Life Technologies Corporation, Carlsbad, CA (US)

(72) Inventors: Sowmi Utiramerur, Pleasanton, CA (US); Dumitru Brinza, Montara, CA (US); Marcin Sikora, Burlingame, CA (US); Christian Koller, Gröbenzell (DE); Earl Hubbell, Palo Alto, CA (US); Chantal Roth, Kallnach (CH); Rajesh Gottimukkala, Fremont, CA (US)

(21) Appl. No.: 18/816,167

(22) Filed: Aug. 27, 2024

Related U.S. Application Data
(63) Continuation of application No. 17/248,083, filed on Jan. 8, 2021, now abandoned.
Application No. 17/248,083 is a continuation of application No. 15/672,865, filed on Aug. 9, 2017, now abandoned.
Application No. 15/672,865 is a division of application No. 13/966,378, filed on Aug. 14, 2013, now abandoned.
(60) Provisional application No. 61/780,124, filed on Mar. 13, 2013.
(60) Provisional application No. 61/755,344, filed on Jan. 22, 2013.
(60) Provisional application No. 61/733,788, filed on Dec. 5, 2012.
(60) Provisional application No. 61/733,799, filed on Dec. 5, 2012.
(60) Provisional application No. 61/682,963, filed on Aug. 14, 2012.

(51) Int. Cl. G16B 30/10; G16B 20/20; G16B 30/00

(52) U.S. Cl. CPC G16B 30/10; G16B 20/20; G16B 30/00

(57) ABSTRACT

Systems and method for determining variants can receive mapped reads and determine a distribution of matched-filter residuals distribution from a plurality of reads at a homopolymer region. The distribution of matched-filter residuals can be fit to uni-modal and bi-modal models. Based on the model that best fits the distribution of matched-filter residuals, the heterozygosity of the sample and the absence or presence of an insertion/deletion in the homopolymer can be determined.

### RELATED APPLICATIONS

This application is a continuation of U.S. patent application Ser. No. 17/248,083 entitled “Systems and Methods for Detecting Homopolymer Insertions/Deletions”, filed on Jan. 8, 2021, which is a continuation of U.S. patent application Ser. No. 15/672,865 entitled “Systems and Methods for Detecting Homopolymer Insertions/Deletions”, filed on Aug. 9, 2017 (now abandoned), which is a divisional of U.S. patent application Ser. No. 13/966,378 entitled “Systems and Methods for Detecting Homopolymer Insertions/Deletions”, filed on Aug. 14, 2013 (now abandoned), which claims priority pursuant to 35 U.S.C. § 119(e) to U.S. Provisional Patent Application Ser. No. 61/682,963, entitled “Systems and Methods for Sequence Identification”, filed on Aug. 14, 2012, U.S. Provisional Patent Application Ser. No. 61/733,799, entitled “Methods for detecting homopolymer insertions/deletions”, filed on Dec. 5, 2012, U.S. Provisional Patent Application Ser. No. 61/780,124, entitled “Methods for detecting homopolymer insertions/deletions”, filed on Mar. 13, 2013, U.S. Provisional Patent Application Ser. No. 61/755,344, entitled “Methods for improved variant calling”, filed on Jan. 22, 2013, U.S. Provisional Patent Application Ser. No. 61/733,788, entitled “Methods for improving model-data confidence in sequencing-by-synthesis”, filed on Dec. 5, 2012, the entirety of which are incorporated herein by reference as if set forth in full.

### FIELD

The present disclosure generally relates to the field of nucleic acid sequencing and, more specifically, relates to systems and methods for identifying genomic variants, including detecting homopolymer insertions/deletions, in nucleic acid sequencing data.

### INTRODUCTION

Upon completion of the Human Genome Project, one focus of the sequencing industry has shifted to finding higher throughput and/or lower cost nucleic acid sequencing technologies, sometimes referred to as “next generation” sequencing (NGS) technologies. In making sequencing higher throughput and/or less expensive, the goal is to make the technology more accessible. These goals can be reached through the use of sequencing platforms and methods that provide sample preparation for samples of significant complexity, sequencing larger numbers of samples in parallel (for example through use of barcodes and multiplex analysis), and/or processing high volumes of information efficiently and completing the analysis in a timely manner. Various methods, such as, for example, sequencing by synthesis, sequencing by hybridization, and sequencing by ligation are evolving to meet these challenges.

Ultra-high throughput nucleic acid sequencing systems incorporating NGS technologies typically produce a large number of short sequence reads. Sequence processing methods should desirably assemble and/or map a large number of reads quickly and efficiently, such as to minimize use of computational resources. For example, data arising from sequencing of a mammalian genome can result in tens or hundreds of millions of reads that typically need to be assembled before they can be further analyzed to determine their biological, diagnostic and/or therapeutic relevance.

Exemplary applications of NGS technologies include, but are not limited to: genomic variant detection, such as insertions/deletions, copy number variations, single nucleotide polymorphisms, etc., genomic resequencing, gene expression analysis and genomic profiling.

Of particular interest are improved systems and methods for identifying genomic variants, including detecting homopolymer insertions/deletions, in homopolymer regions, and thereby detecting heterozygosity and improving accuracy of nucleic acid sequencing. Recent advances in genotyping technologies have resulted in a better understanding of common human sequence variation, which has led to the identification of many novel genetic determinants of complex traits/diseases. Nevertheless, despite these successes, much of the genetic component of these traits/diseases remains incomplete. Although there may be many undiscovered polymorphisms associated with complex traits/diseases, the “common-disease common-variant” paradigm may not provide a complete picture.

From the foregoing it will be appreciated that a need exists for systems and methods that can detect insertions/deletions in homopolymer regions in nucleic acid sequencing data.

### BRIEF DESCRIPTION OF DRAWINGS

For a more complete understanding of the principles disclosed herein, and the advantages thereof, reference is now made to the following descriptions taken in conjunction with the accompanying drawings, in which:

FIG. 1 is a block diagram that illustrates an exemplary computer system, in accordance with various embodiments.

FIG. 2 is a schematic diagram of an exemplary system for reconstructing a nucleic acid sequence, in accordance with various embodiments.

FIG. 3 is a flow diagram illustrating an exemplary method of detecting insertions/deletions, in accordance with various embodiments.

FIG. 4 is a schematic diagram of an exemplary variant calling system, in accordance with various embodiments.

FIG. 5 shows an observed distribution of matched-filter residuals in the event of a homozygous deletion of a 3-mer in Reference to a 2-mer in the sample.

FIG. 6 shows an observed distribution of matched-filter residuals in the event of a heterozygous deletion of a 4-mer in Reference to a 3-mer in the sample.

FIG. 7 shows an observed distribution of matched-filter residuals in the event of a heterozygous two base deletion of a 5-mer in Reference to a 3-mer in the sample.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DESCRIPTION OF VARIOUS EMBODIMENTS

Embodiments of systems and methods for identifying genomic variants, including detecting homopolymer insertions/deletions, are described herein.

The section headings used herein are for organizational purposes only and are not to be construed as limiting the described subject matter in any way.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless described otherwise, all technical and scientific terms used herein have a meaning as is commonly understood by one of ordinary skill in the art to which the various embodiments described herein belongs.

It will be appreciated that there is an implied “about” prior to the temperatures, concentrations, times, number of bases, coverage, etc., discussed in the present teachings, such that slight and insubstantial deviations are within the scope of the present teachings. In this application, the use of the singular includes the plural unless specifically stated otherwise. Also, the use of “comprise”, “comprises”, “comprising”, “contain”, “contains”, “containing”, “include”, “includes”, and “including” are not intended to be limiting. It is to be understood that both the foregoing general description and the following detailed description are exemplary and explanatory only and are not restrictive of the present teachings.

Further, unless otherwise required by context, singular terms shall include pluralities and plural terms shall include the singular. Generally, nomenclatures utilized in connection with, and techniques of, cell and tissue culture, molecular biology, and protein and oligo—or polynucleotide chemistry and hybridization described herein are those well known and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the instant specification. See, e.g., Sambrook et al., Molecular Cloning: A Laboratory Manual (Third ed., Cold Spring Harbor Laboratory Press, Cold Spring Harbor, N.Y. 2000). The nomenclatures utilized in connection with, and the laboratory procedures and techniques described herein are those well known and commonly used in the art.

As used herein, “a” or “an” also may refer to “at least one” or “one or more.”

A “system” sets forth a set of components, real or abstract, comprising a whole where each component interacts with or is related to at least one other component within the whole.

A “biomolecule” may refer to any molecule that is produced by a biological organism, including large polymeric molecules such as proteins, polysaccharides, lipids, and nucleic acids (DNA and RNA) as well as small molecules such as primary metabolites, secondary metabolites, and other natural products.

The phrase “next generation sequencing” or NGS refers to sequencing technologies having increased throughput as compared to traditional Sanger—and capillary electrophoresis-based approaches, for example with the ability to generate hundreds of thousands of relatively small sequence reads at a time. Some examples of next generation sequencing techniques include, but are not limited to, sequencing by synthesis, sequencing by ligation, and sequencing by hybridization. More specifically, the Ion Personal Genome Machine® (PGM™) of Life Technologies Corp. provides massively parallel sequencing with enhanced accuracy. Aspects of the Ion PGM™ Sequencer and associated workflows, protocols, chemistries, etc., are described in more detail in U.S. Patent Application Publication No. 2009/0127589 and No. 2009/0026082, the entirety of each of these applications being incorporated herein by reference.

The phrase “sequencing run” refers to any step or portion of a sequencing experiment performed to determine some information relating to at least one biomolecule (e.g., nucleic acid molecule).

The phase “base space” refers to a representation of the sequence of nucleotides. The phase “flow space” refers to a representation of the incorporation event or non-incorporation event for a particular nucleotide flow. For example, flow space can be a series of zeros and ones representing a nucleotide incorporation event (a one, “1”) or a non-incorporation event (a zero, “0”) for that particular nucleotide flow. It should be understood that zeros and ones are convenient representations of a non-incorporation event and a nucleotide incorporation event; however, any other symbol or designation could be used alternatively to represent and/or identify these events and non-events.

DNA (deoxyribonucleic acid) is a chain of nucleotides comprising 4 types of nucleotides; A (adenine), T (thymine), C (cytosine), and G (guanine), and that RNA (ribonucleic acid) comprising 4 types of nucleotides; A, U (uracil), G, and C. Certain pairs of nucleotides specifically bind to one another in a complementary fashion (called complementary base pairing). That is, adenine (A) can pair with thymine (T) (in the case of RNA, however, adeninem (A) can pair with uracil (U)), and cytosine (C) can pair with guanine (G). When a first nucleic acid strand binds to a second nucleic acid strand made up of nucleotides that are complementary to those in the first strand, the two strands bind to form a double strand. As used herein, “nucleic acid sequencing data,” “nucleic acid sequencing information,” “nucleic acid sequence,” “genomic sequence,” “genetic sequence,” or “fragment sequence,” or “nucleic acid sequencing read” denotes any information or data that is indicative of the order of the nucleotide bases (e.g., adenine, guanine, cytosine, and thymine/uracil) in a molecule (e.g., whole genome, whole transcriptome, exome, oligonucleotide, polynucleotide, fragment, etc.) of DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion-or pH-based detection systems, electronic signature-based systems, etc.

A “polynucleotide”, “nucleic acid”, or “oligonucleotide” refers to a linear polymer of nucleosides (including deoxyribonucleosides, ribonucleosides, or analogs thereof) joined by internucleosidic linkages. Typically, a polynucleotide comprises at least three nucleosides. Usually oligonucleotides range in size from a few monomeric units, e.g., 3-4, to several hundreds of monomeric units. Whenever a polynucleotide such as an oligonucleotide is represented by a sequence of letters, such as “ATGCCTG,” it will be understood that the nucleotides are in 5′->3 ′ order from left to right and that “A” denotes deoxyadenosine, “C” denotes deoxycytidine, “G” denotes deoxyguanosine, and “T” denotes thymidine, unless otherwise noted. The letters A, C, G, and T may be used to refer to the bases themselves, to nucleosides, or to nucleotides comprising the bases, as is standard in the art.

The techniques of “paired-end,” “pairwise,” “paired tag,” or “mate pair” sequencing are generally known in the art of molecular biology (Siegel A. F. et al., Genomics. 2000, 68: 237-246; Roach J. C. et al., Genomics. 1995, 26: 345-353). These sequencing techniques provide for the determination of multiple “reads” of sequence information from different regions on a polynucleotide strand. Typically, the distance, such as an insert region or a gap, between the reads or other information regarding a relationship between the reads is known or can be approximated. In some situations, these sequencing techniques provide more information than does sequencing stretches of nucleic acid sequences in a random fashion. With the use of appropriate software tools for the assembly of sequence information (e.g., Millikin S. C. et al., Genome Res. 2003, 13: 81-90; Kent, W.J. et al., Genome Res. 2001, 11:1541-8) it is possible to make use of the knowledge that the “paired-end,” “pairwise,” “paired tag” or “mate pair” sequences are not completely random, but are known or anticipated to occur some distance apart and/or to have some other relationship, and are therefore linked or paired with respect to their position within the genome. This information can aid in the assembly of whole nucleic acid sequences into a consensus sequence.

### Computer-Implemented System

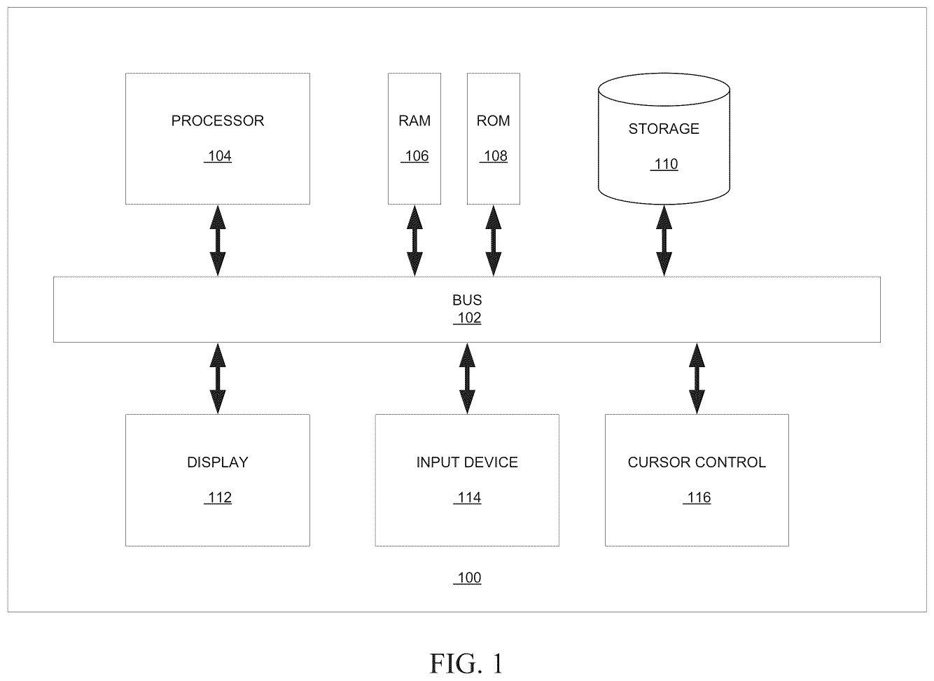

FIG. 1 is a block diagram that illustrates a computer system 100, upon which embodiments of the present teachings may be implemented. In various embodiments, computer system 100 can include a bus 102 or other communication mechanism for communicating information, and a processor 104 coupled with bus 102 for processing information. In various embodiments, computer system 100 can also include a memory 106, which can be a random access memory (RAM) or other dynamic storage device, coupled to bus 102 for determining base calls, and instructions to be executed by processor 104. Memory 106 also can be used for storing temporary variables or other intermediate information during execution of instructions to be executed by processor 104. In various embodiments, computer system 100 can further include a read only memory (ROM) 108 or other static storage device coupled to bus 102 for storing static information and instructions for processor 104. A storage device 110, such as a magnetic disk or optical disk, can be provided and coupled to bus 102 for storing information and instructions.

In various embodiments, computer system 100 can be coupled via bus 102 to a display 112, such as a cathode ray tube (CRT) or liquid crystal display (LCD), for displaying information to a computer user. An input device 114, including alphanumeric and other keys, can be coupled to bus 102 for communicating information and command selections to processor 104. Another type of user input device is a cursor control 116, such as a mouse, a trackball or cursor direction keys for communicating direction information and command selections to processor 104 and for controlling cursor movement on display 112. This input device typically has two degrees of freedom in two axes, a first axis (i.e., x) and a second axis (i.e., y), that allows the device to specify positions in a plane.

A computer system 100 can perform the present teachings. Consistent with certain implementations of the present teachings, results can be provided by computer system 100 in response to processor 104 executing one or more sequences of one or more instructions contained in memory 106. Such instructions can be read into memory 106 from another computer-readable medium, such as storage device 110. Execution of the sequences of instructions contained in memory 106 can cause processor 104 to perform the processes described herein. Alternatively hard-wired circuitry can be used in place of or in combination with software instructions to implement the present teachings. Thus implementations of the present teachings are not limited to any specific combination of hardware circuitry and software.

The term “computer-readable medium” as used herein refers to any media that participates in providing instructions to processor 104 for execution. Such a medium can take many forms, including but not limited to, non-volatile media, volatile media, and transmission media. Examples of non-volatile media can include, but are not limited to, optical or magnetic disks, such as storage device 110. Examples of volatile media can include, but are not limited to, dynamic memory, such as memory 106. Examples of transmission media can include, but are not limited to, coaxial cables, copper wire, and fiber optics, including the wires that comprise bus 102.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In accordance with various embodiments, instructions configured to be executed by a processor to perform a method are stored on a computer-readable medium. The computer-readable medium can be a device that stores digital information. For example, a computer-readable medium includes a compact disc read-only memory (CD-ROM) as is known in the art for storing software. The computer-readable medium is accessed by a processor suitable for executing instructions configured to be executed.

### Nucleic Acid Sequencing Platforms

Nucleic acid sequence data can be generated using various techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronic signature-based systems, etc.

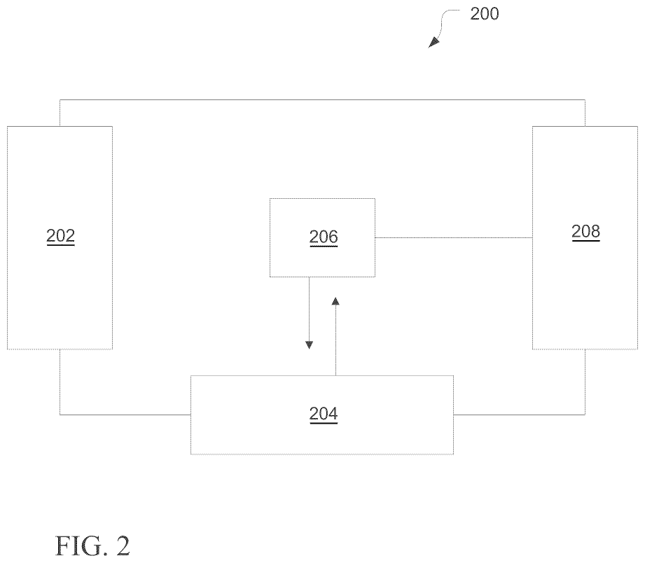

Various embodiments of nucleic acid sequencing platforms, such as a nucleic acid sequencer, can include components as displayed in the block diagram of FIG. 2. According to various embodiments, sequencing instrument 200 can include a fluidic delivery and control unit 202, a sample processing unit 204, a signal detection unit 206, and a data acquisition, analysis and control unit 208. Various embodiments of instrumentation, reagents, libraries and methods used for next generation sequencing are described in U.S. Patent Application Publication No. 2009/0127589 and No. 2009/0026082, which are incorporated herein by reference in their entirety. Various embodiments of instrument 200 can provide for automated sequencing that can be used to gather sequence information from a plurality of sequences in parallel, such as substantially simultaneously.

In various embodiments, the fluidics delivery and control unit 202 can include reagent delivery system. The reagent delivery system can include a reagent reservoir for the storage of various reagents. The reagents can include RNA-based primers, forward/reverse DNA primers, oligonucleotide mixtures for ligation sequencing, nucleotide mixtures for sequencing-by-synthesis, optional ECC oligonucleotide mixtures, buffers, wash reagents, blocking reagent, stripping reagents, and the like. Additionally, the reagent delivery system can include a pipetting system or a continuous flow system which connects the sample processing unit with the reagent reservoir. In various embodiments, the reagents may comprise nucleotide species delivered according to a pre-determined ordering as described in Hubbell et al., U.S. Patent Application Publication No. 2012/0264621, published Oct. 18, 2012, which is incorporated by reference herein in its entirety.

In various embodiments, the sample processing unit 204 can include a sample chamber, such as flow cell, a substrate, a micro-array, a multi-well tray, or the like. The sample processing unit 204 can include multiple lanes, multiple channels, multiple wells, or other means of processing multiple sample sets substantially simultaneously. Additionally, the sample processing unit can include multiple sample chambers to enable processing of multiple runs simultaneously. In particular embodiments, the system can perform signal detection on one sample chamber while substantially simultaneously processing another sample chamber. Additionally, the sample processing unit can include an automation system for moving or manipulating the sample chamber.

In various embodiments, the signal detection unit 206 can include an imaging or detection sensor. For example, the imaging or detection sensor can include a CCD, a CMOS, an ion or chemical sensor, such as an ion sensitive layer overlying a CMOS or FET, a current or voltage detector, or the like. The signal detection unit 206 can include an excitation system to cause a probe, such as a fluorescent dye, to emit a signal. The excitation system can include an illumination source, such as arc lamp, a laser, a light emitting diode (LED), or the like. In particular embodiments, the signal detection unit 206 can include optics for the transmission of light from an illumination source to the sample or from the sample to the imaging or detection sensor. Alternatively, the signal detection unit 206 may provide for electronic or non-photon based methods for detection and consequently not include an illumination source. In various embodiments, electronic-based signal detection may occur when a detectable signal or species is produced during a sequencing reaction. For example, a signal can be produced by the interaction of a released byproduct or moiety, such as a released ion, such as a hydrogen ion, interacting with an ion or chemical sensitive layer. In other embodiments a detectable signal may arise as a result of an enzymatic cascade such as used in pyrosequencing (see, for example, U.S. Patent Application Publication No. 2009/0325145, the entirety of which being incorporated herein by reference) where pyrophosphate is generated through base incorporation by a polymerase which further reacts with ATP sulfurylase to generate ATP in the presence of adenosine 5′ phosphosulfate wherein the ATP generated may be consumed in a luciferase mediated reaction to generate a chemiluminescent signal. In another example, changes in an electrical current can be detected as a nucleic acid passes through a nanopore without the need for an illumination source.

In various embodiments, a data acquisition analysis and control unit 208 can monitor various system parameters. The system parameters can include temperature of various portions of instrument 200, such as sample processing unit or reagent reservoirs, volumes of various reagents, the status of various system subcomponents, such as a manipulator, a stepper motor, a pump, or the like, or any combination thereof.

It will be appreciated by one skilled in the art that various embodiments of instrument 200 can be used to practice variety of sequencing methods including ligation-based methods, sequencing by synthesis, single molecule methods, nanopore sequencing, and other sequencing techniques.

In various embodiments, sequencing data may be obtained using sequencing-by-synthesis. A template polynucleotide strand can be exposed to a series of flows of nucleotide species in the presence of a polymerase. When the nucleotide species of the flow complements the next base of the template polynucleotide strand, the polymerase can incorporate the nucleotide into a complementary polynucleotide strand.

In a particular embodiment, the incorporation can be detected by measuring a change in an electrical property of a field effect transistor proximate to the extending complementary polynucleotide strand. The change in the electrical property can be in response to a change in ion concentration in the vicinity of the extending complementary polynucleotide strand and field effect transistor, such as due to a release of a hydrogen ion when the nucleotide is incorporated into the complementary polynucleotide strand by the polymerase. In another particular embodiment, the incorporation can be detected by measuring an intensity of photons generated by a chemiluminescent reaction triggered by the incorporation event.

When the template polynucleotide strand includes a homopolymer stretch, the polymerase can incorporate multiple complementary nucleotides during a single flow. The measured value, such as change in electrical property or intensity of photons, can be proportional to the number of nucleotide incorporations during the flow and therefore can be proportional to the length of the homopolymer stretch.

In various embodiments, the sequencing instrument 200 can determine the sequence of a nucleic acid, such as a polynucleotide or an oligonucleotide. The nucleic acid can include DNA or RNA, and can be single stranded, such as ssDNA and RNA, or double stranded, such as dsDNA or a RNA/cDNA pair. In various embodiments, the nucleic acid can include or be derived from a fragment library, a mate pair library, a ChIP fragment, or the like. In particular embodiments, the sequencing instrument 200 can obtain the sequence information from a single nucleic acid molecule or from a group of substantially identical nucleic acid molecules.

In various embodiments, sequencing instrument 200 can output nucleic acid sequencing read data in a variety of different output data file types/formats, including, but not limited to: *bam, *.fasta, *.csfasta, *seq.txt, *qseq.txt, *.fastq, *.sff, *prb.txt, *.sms, *srs and/or *.qv.

### System and Methods for Identifying Sequence Variation

Identification of sequence variants including single nucleotide polymorphism (SNPs), insertions, and deletions is an important application of next generation sequencing technologies. In various embodiments, the approach/technology implemented during sequencing can influence the accuracy of variant identification. Likewise, the analytical approach used during sequence resolution and alignment can affect the overall quality of the data. For example, in certain embodiments, base space alignment methodologies can misplace or miscall insertions or deletions in the alignment of flow space reads generated using sequencing by synthesis platforms such as the Ion PGM™.

### Example 1: (An Example That May Occur With Increased Frequency)

| | | |
|---|---|---|
| AAAAAATTTTT | ← | reference |
| AAAAATTTTTT | ← | read1 |
| AAAAAATTTTT | ← | read2 |
| AAAAATTTTTT | ← | read3 |
| AAAAAATTTTT | ← | read4 |

### Example 2: (Another Miss-Aligned Example)

| | | |
|---|---|---|
| AAAAAACTTTTT | ← | reference |
| AAAAAC--TTTT | ← | read1 |
| AAAAAACTTTTT | ← | read2 |
| AAAAAC--TTTT | ← | read3 |
| AAAAAACTTTTT | ← | read4 |

In the examples above the more likely alignment (explanation) of alignment for reads 1 and 3 may be as follows:

| | | |
|---|---|---|
| AAAAAA-TTTTT | ← | reference |
| AAAAA-TTTTTT | ← | read1 |
| AAAAA-TTTTTT | ← | read3 |

In various embodiments, although the alignment above may be more likely to be true, it is not necessarily always the correct one. For example, an A→T SNP at the middle position as indicated may not be as rare as expected. Using flow space alignment and pileup to select the above alignments, overlooking or misidentifying such types of alignments may occur. In various instances such as the two alignments shown above two forms (mismatch vs undercall+overcall) may be statistically in the same order of magnitude. In such instances, it may be difficult or impractical for an automated sequence or fragment alignment routine to select or identify the most accurate or true candidate. For example, the likelihood of a mismatch occurring may be approximately 0.5%, and the chance of undercall followed by overcall might be large.

From the foregoing discussion it will be appreciated that during sequence analysis a portion of the alignment may be of lower quality. It is therefore not uncommon that the selected analysis path may lead to an incorrect or lower quality result.

In many cases, however, irrespective of the sequence alignment algorithm used for poorly aligned base reads, it may be expected that the bases are not far from their correct positioning/alignments. This can be observed for single bases as well as multiple bases in an exemplary read.

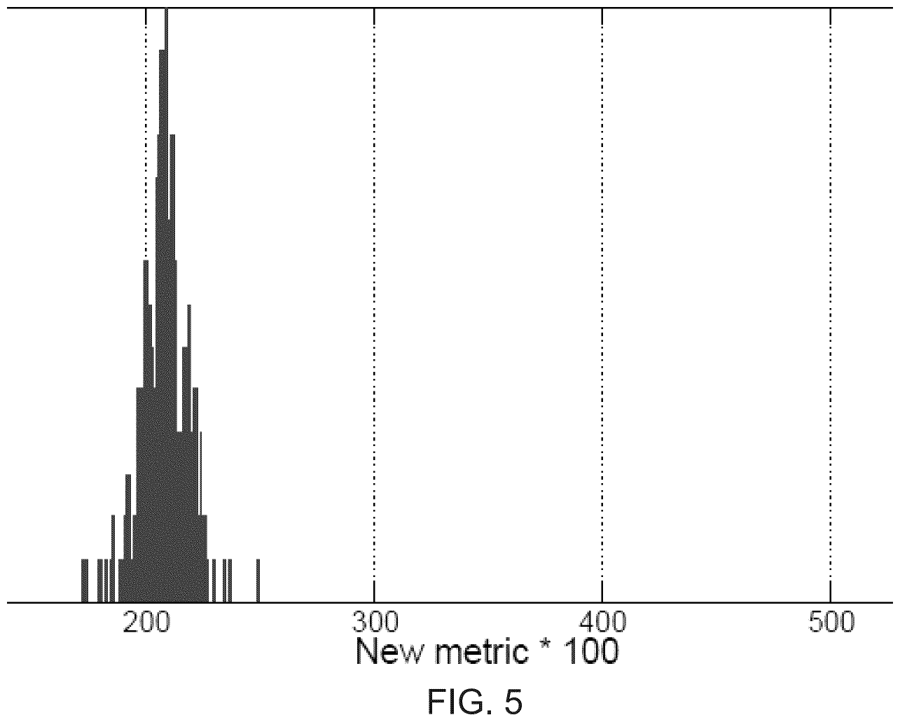

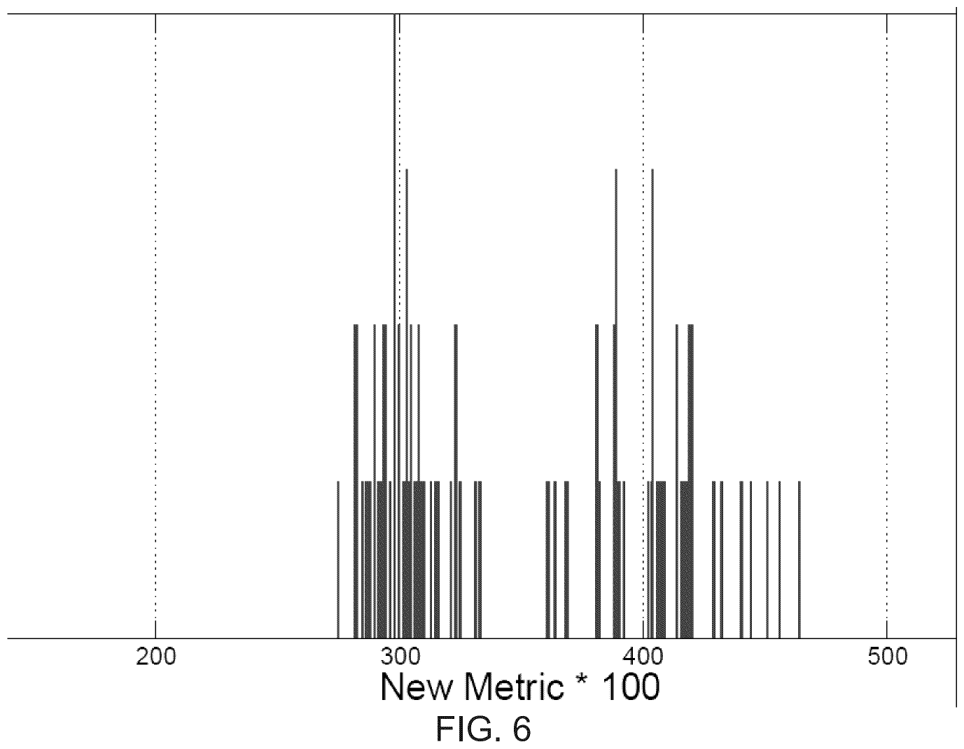

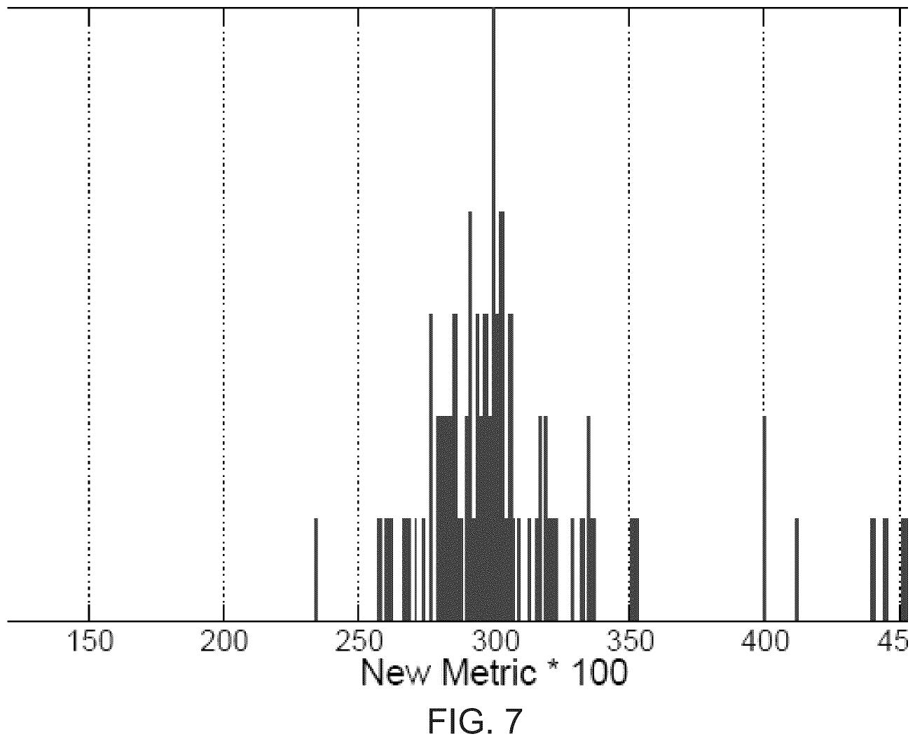

One improved method for basecalling may include applying a Bayesian based peak detection approach to detect insertions/deletions and determine heterozygosity in homopolymer regions. In various embodiments, certain residuals associated with predictive base calling can be shown to have Gaussian distribution. See, for example, FIG. 5. Homozygous homopolymer regions tend to be uni-modal. See, for example, FIG. 5. Heterozygous homopolymer regions tend to be bi-modal. See, for example, FIGS. 6 and 7. Thus, predicting the number of peaks can differentiate heterozygous positions from positions with a high degree of noise in residuals associated with predictive base calling.

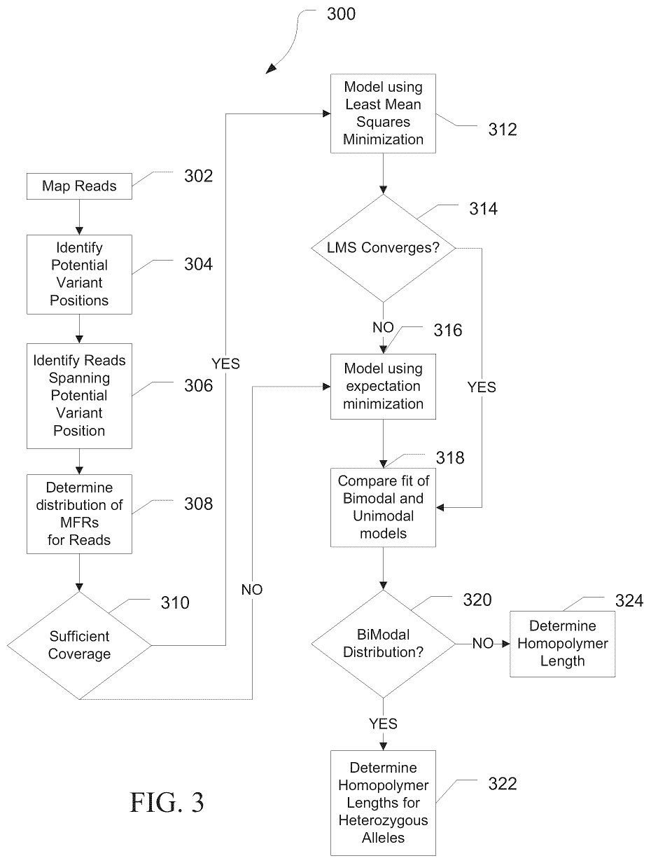

FIG. 3 is an exemplary flow diagram showing a method 300 for identifying variants, including detecting homopolymer insertions/deletions, in nucleic acid sequence reads, in accordance with various embodiments. At 302, reads can be mapped to a reference genome. Various algorithms are known in the art for mapping reads to a reference genome. In particular embodiments, the mapping to the reference genome can be performed in base space after the reads are converted from flow space to base space.

At 304, the mapped reads can be used to identify potential variant positions (e.g., candidate indel variants). Potential variant positions are positions where some number of reads that map to a position have a sequence that does not match the reference sequence. In particular embodiments, the homopolymer length in at least a portion of the reads that map to the homopolymer region can be greater than or less than the homopolymer length of the reference sequence.

At 306, the reads spanning the potential variant position can be identified, and at 308, the distribution of MFRs (matched-filter residuals) for the homopolymer region can be determined for the reads that span the potential variant position.

More specifically, in an exemplary embodiment, each candidate indel may be classified as either a homopolymer indel or a non-homopolymer indel, where homopolymer indels are either an insertion or deletion of one or more of the same base as the stretch of homopolymers in the reference sequence (e.g., “TTTT”->“TTTTT”), and the homopolymer indels may be evaluated using a matched-filter residual computed for two hypotheses: Hypothesis one, which contains the same number of bases or homopolymer length as the reference sequence, and Hypothesis two, which contains either more or less number of bases compared to the reference homopolymer length depending on the size of the indel candidate being evaluated.

In an exemplary embodiment, the matched-filter residual may be calculated using the following equation:

$$Δ = \frac{\sum ( {y}_{i} - {x}_{A , i} ) ( {x}_{B , i} - {x}_{A , i} )}{\sum {( {x}_{B , i} - {x}_{A , i} )}^{2}}$$

where y =(y₁, . . . , y_{N}) represents normalized measurements, x_{A}=(x_{A,1}, . . . , x_{A,N}) represents a predicted signal generated by a phasing model for a read r_{A} using available phasing parameters, and x_{B}=(x_{B,1}, . . . , x_{B,N}) represents a predicted signal generated by a phasing model for a read r_{B} using available phasing parameters, c =(c₁, . . . , c_{L}, h_{c}, c_{R}, . . . , c_{K}) represents a read sequence called by a base caller (with an emphasis on homopolymer h_{c}, a possible variant indel variant site), r_{A}=(c₁, . . . , c_{L}, h_{A}, c_{R}, . . . , c_{K}) represents a modified read sequence where called hompolymer h_{c} is replaced by reference homopolymer h_{A} of same nucleotide but possibly different length, and r_{B}=(c₁, . . . , c_{L}, h_{B}, c_{R}, . . . , c_{K}) represents a modified read sequence where called homopolymer h_{c} is replaced by homopolymer h_{B}, one base longer than h_{A}, where K represents a number of called bases and N represents a number of flows. The matched-filter residual represents the state-weighted deviation between measurements y and prediction x_{A} that can be attributed to the difference between h_{c} and h_{A}.

In various embodiments, normalization of measurements, generation of predicted signals using a phasing model and/or phasing parameters, and calling of bases may be performed as described in one or more of Davey et al., U.S. Pat. Appl. Publ. No. 2012/0109598, published May 3, 2012, Sikora et al., U.S. patent application Ser. No. 13/588,408, filed Aug. 17, 2012, and in Sikora et al., U.S. patent application Ser. No. 13/645,058, filed Oct. 4, 2012, which are all incorporated by reference herein in their entirety.

According to an embodiment, there is provided a method for determining a presence of an indel variant in a reference homopolymer, comprising: (1) retrieving a called sequence c and normalized measurements y for a specific read covering a reference homopolymer h_{A}; (2) establishing location h_{c} within sequence c that aligned to reference homopolymer h_{A} based on alignment results; (3) creating modified read sequences r_{A} and r_{B}, by substituting h_{c} with h_{A} and h_{B}, where h_{B} is one base longer than h_{A}; (4) generating a predicted signal x_{A} for r_{A} and a predicted signal x_{B} for r_{B} using one or more phasing parameters and a pre-determine ordering of nucleotide species flows; and (5) calculating a state-weighted deviation between measurements y and prediction x_{A} that can be attributed to difference between h_{c} and h_{A}.

For example, if

c = TCAGT CCC GA
r_{A} = TCAGT CCCC GA
r_{B} = TCAGT CCCCC GA

and
x_{A}=(0.99, 0.00, 1.00, 0.02, 0.02, 0.96, 0.07, 0.97, 0.95, 3.77, 0.01, 0.94, 0.96, 0.02, 0.04, 0.01, 0.02, 0.08, 0.03, 0.02, 0.00)
x_{B}=(0.99, 0.00, 1.00, 0.02, 0.02, 0.96, 0.09, 0.97, 0.95, 4.72, 0.01, 0.94, 0.96, 0.02, 0.05, 0.01, 0.02, 0.10, 0.03, 0.03, 0.00)
y =(1.00, 0.04, 0.96, 0.03, 0.02, 0.94, 0.05, 0.98, 1.02, 2.88, 0.00, 1.00, 0.98, 0.02, 0.04, 0.01, 0.02, 0.09, 0.06, 0.06, 0.01)
   then

$$Δ = - 0.935 \; 6 .$$

In an exemplary embodiment, such deltas may be computed for each read that spans the candidate indel position and a distribution of the observed deltas may be generated. In an example, the value of delta may be multiplied by 100 and rounded to nearest integer value to retain only the two significant digits. A suitable estimator module may then evaluate the distribution of deltas to determine if it is uni-modal or bi-modal. A goodness-of-fit test may be performed using the observed distribution of deltas against the expected uni-modal and bi-modal Gaussian distributions, and the zygosity may be determined by the best goodness-of-fit value.

At 310, a determination can be made as to the sufficiency of the coverage for the homopolymer region. In various embodiments, the determination can be made based on a predetermined cutoff, such as determining there is sufficient coverage when the number of reads spanning the homopolymer region is at least about 100, such as at least about 200, even at least about 300. In various embodiments, the cutoff can be determined based on the homopolymer length, such as the homopolymer length in the reference sequence or the average homopolymer length of the reads. For example, longer homopolymer regions can have a higher cutoff than a shorter homopolymer region. In other embodiments, sufficiency of coverage can be determined based on characteristics of the distribution of the matched-filter residuals, such as the range of matched-filter residuals, the amount of variability in the frequencies for similar matched-filter residuals, and the like.

When there is sufficient coverage, the distribution of matched-filter residuals can be fit to uni-modal and bi-modal distributions using least mean squares minimization, as illustrated at 312. The uni-modal distribution can include a single Gaussian distribution represented by a single peak and the bi-modal distribution can include two Gaussian distributions represented by two peaks. Depending on the separation between the means and the variance of the two Gaussian distributions, the two curves may overlap resulting in a composite curve.

At 314, a determination can be made if the least mean squares minimization converged. When the least means squares minimization fails to converge for both the uni-modal and bi-modal Gaussian distribution models, or when there is insufficient coverage from 310, the distribution of matched-filter residuals can be fit to uni-modal and bi-modal Gaussian models using expectation minimization, as illustrated at 316.

At 318, either from 312 when the least mean squares minimization converges for at least one of the uni-modal or bi-modal Gaussian models, or from 316 based on the expectation minimization results, the fit of the uni-modal and bi-modal Gaussian models can be compared. At 320, a determination can be made as to if the distribution of matched-filter residuals is bi-modal based on the comparison of fits to the uni-modal and bi-modal Gaussian models.

When the distribution of matched-filter residuals values is bi-modal, such as when only the bi-modal Gaussian model converged for least mean squares minimization or the fit to the bi-modal Gaussian model is statistically more significant that the fit to the uni-modal Gaussian model, at 322, the sample can be determined to be heterozygous and lengths of the homopolymer region for the two alleles can be determined from the centers of the two peaks in the bi-modal Gaussian model. In various embodiments, a relative abundance of the two alleles present in the sample can be determined from the magnitude of the two peaks in the bi-modal Gaussian model.

Alternatively, when the distribution of matched-filter residuals is uni-modal, such as when only the uni-modal Gaussian model converged for least mean squares minimization or the fit to the uni-modal Gaussian model is statistically more significant, at 324, the length of the homopolymer region can be determined based on the center of the peak in the uni-modal Gaussian model.

FIG. 5 shows an observed distribution of matched-filter residuals in the event of a homozygous deletion of a 3-mer in Reference to a 2-mer in the sample.

FIG. 6 shows an observed distribution of matched-filter residuals in the event of a heterozygous deletion of a 4-mer in Reference to a 3-mer in the sample.

FIG. 7 shows an observed distribution of matched-filter residuals in the event of a heterozygous two base deletion of a 5-mer in Reference to a 3-mer in the sample.

According to an exemplary embodiment, there is provided a system for identifying variants, comprising: a mapping component configured to use a processor to map a plurality of reads to a reference genome; and a variant calling component communicatively connected with the mapping component, the variant calling component configured to determine a distribution of base calling residuals based on measured and model-predicted values for a homopolymer region from a plurality of reads from a sample, the reads spanning the homopolymer region.

In such a system, the variant calling component may be further configured to fit the base calling residuals distribution to a uni-modal model and a bi-modal model to determine a best-fit model. The uni-modal model may be a uni-modal Gaussian model and the bi-modal model may be a bi-modal Gaussian model. The uni-modal Gaussian model may include one Gaussian distribution and the bi-modal Gaussian model may include first and second Gaussian distributions. The variant calling component may be further configured to, when the best-fit model is the bi-modal Gaussian distribution, identify the sample as heterozygous. The variant calling component may be further configured to, when the best-fit model is the bi-modal Gaussian distribution, determine a first homopolymer length and a second homopolymer length based on centers of the first and second Gaussian distributions. The variant calling component may be further configured to, when the best-fit model is the bi-modal Gaussian distribution, output the first and second homopolymer lengths. The model-predicted values may be determined from a predictive model of phasing effects. The base calling residuals may be matched-filter residual representing state-weighted deviation between measurements y and a prediction x_{A} that can be attributed to the difference between h_{c} and h_{A}, where y =(y₁, . . . , y_{N}) represents normalized measurements, x_{A}=(x_{A,1}, . . . , x_{A,N}) represents a predicted signal generated by a phasing model for a read r_{A} using available phasing parameters, and x_{B}=(x_{B,1}, . . . , x_{B,N}) represents a predicted signal generated by a phasing model for a read r_{B} using available phasing parameters, c =(c₁, . . . , c_{L}, h_{c}, c_{R}, . . . , c_{K}) represents a read sequence called by a base caller (with an emphasis on homopolymer h_{c}, a possible variant indel variant site), r_{A}=(c₁, . . . , c_{L}, h_{A}, c_{R}, . . . , c_{K}) represents a modified read sequence where called hompolymer h_{c} is replaced by reference homopolymer h_{A} of same nucleotide but possibly different length, and r_{B}=(c₁, . . . , c_{L}, h_{B}, c_{R}, . . . , c_{K}) represents a modified read sequence where called homopolymer h_{c} is replaced by homopolymer h_{B}, one base longer than h_{A}, where K represents a number of called bases and N represents a number of flows. The variant calling component may be further configured to, when the best-fit model is the uni-modal Gaussian distribution: identify the sample as not heterozygous; determine the homopolymer length a based on a center of the Gaussian distribution; and output the homopolymer length.

According to an exemplary embodiment, there is provided a method for determining a presence or absence of an insertion/deletion variant in a reference homopolymer, comprising: obtaining sequencing data relating to a plurality of template polynucleotide strands disposed in a sensor array, the template polynucleotide strands having been exposed to a series of flows of nucleotide species; generating one or more preliminary sequences of called bases by performing a preliminary base calling for at least some of the plurality of template polynucleotide strands using the sequencing data; identifying one or more candidate variant sequences in the one or more preliminary sequences of called bases by mapping the one or more preliminary sequences of called bases against a reference genome; and calling one or more variants using a distribution of base calling residuals based on measured and model-predicted values, including retrieving, for at least one of the one or more candidate variant sequences, a called sequence c and corresponding normalized measurements y from the sequencing data covering a reference homopolymer h_{A}.

Such a method may comprise establishing a location h_{c} within sequence c that aligned to reference homopolymer h_{A} based on alignment results. Such a method may comprise creating modified read sequences r_{A} and r_{B}, by substituting h_{c} with h_{A} and h_{B}, where h_{B} is one base longer than h_{A}. Such a method may comprise generating a model-predicted signal x_{A} for r_{A} using one or more phasing parameters and a pre-determined ordering of nucleotide species flows. Such a method may comprise generating a model-predicted signal x_{B} for r_{B} using one or more phasing parameters and a pre-determined ordering of nucleotide species flows. Such a method may comprise calculating a state-weighted deviation between measurements y and prediction x_{A} that can be attributed to difference between h_{c} and h_{A}.

According to an exemplary embodiment, there is provided a non-transitory machine-readable storage medium comprising instructions which, when executed by a processor, cause the processor to perform a method for determining a presence or absence of an insertion/deletion variant in a reference homopolymer comprising: receiving sequencing data relating to a plurality of template polynucleotide strands disposed in a sensor array, the template polynucleotide strands having been exposed to a series of flows of nucleotide species; generating one or more preliminary sequences of called bases by performing a preliminary base calling for at least some of the plurality of template polynucleotide strands using the sequencing data; identifying one or more candidate variant sequences in the one or more preliminary sequences of called bases by mapping the one or more preliminary sequences of called bases against a reference genome; and calling one or more variants using a distribution of base calling residuals based on measured and model-predicted values, including retrieving, for at least one of the one or more candidate variant sequences, a called sequence c and corresponding normalized measurements y from the sequencing data covering a reference homopolymer h_{A}. The method may further comprise: establishing a location h_{c} within sequence c that aligned to reference homopolymer h_{A} based on alignment results; creating modified read sequences r_{A} and r_{B}, by substituting h_{c} with h_{A} and h_{B}, where h_{B} is one base longer than h_{A}; generating a model-predicted signal x_{A} for r_{A} using one or more phasing parameters and a pre-determined ordering of nucleotide species flows; generating a model-predicted signal x_{B} for r_{B} using one or more phasing parameters and a pre-determined ordering of nucleotide species flows; and calculating a state-weighted deviation between measurements y and prediction x_{A} that can be attributed to difference between h_{c} and h_{A}.

According to an exemplary embodiment, there is provided a system, including: a machine-readable memory; and a processor configured to execute machine-readable instructions, which, when executed by the processor, cause the system to perform a method for determining a presence or absence of an insertion/deletion variant in a reference homopolymer comprising: receiving sequencing data relating to a plurality of template polynucleotide strands disposed in a sensor array, the template polynucleotide strands having been exposed to a series of flows of nucleotide species; generating one or more preliminary sequences of called bases by performing a preliminary base calling for at least some of the plurality of template polynucleotide strands using the sequencing data; identifying one or more candidate variant sequences in the one or more preliminary sequences of called bases by mapping the one or more preliminary sequences of called bases against a reference genome; and calling one or more variants using a distribution of base calling residuals based on measured and model-predicted values, including retrieving, for at least one of the one or more candidate variant sequences, a called sequence c and corresponding normalized measurements y from the sequencing data covering a reference homopolymer h_{A}. The method may further comprise: establishing a location h_{c} within sequence c that aligned to reference homopolymer h_{A} based on alignment results; creating modified read sequences r_{A} and r_{B}, by substituting h_{c} with h_{A} and h_{B}, where h_{B} is one base longer than h_{A}; generating a model-predicted signal x_{A} for r_{A} using one or more phasing parameters and a pre-determined ordering of nucleotide species flows; generating a model-predicted signal x_{B} for r_{B} using one or more phasing parameters and a pre-determined ordering of nucleotide species flows; and calculating a state-weighted deviation between measurements y and prediction x_{A} that can be attributed to difference between h_{c} and h_{A}.

In various embodiments, a probability model can be used to determine the probability of each base call being over-called or under-called relative to resolution of the entire read. For example, for a base there can be a measurement that describes how surrounding raw signals that support the call. In an embodiment, an intensity value corresponding to a base call at position j in the highest scored predicted sequence is replaced with the maximum between probabilities of that base being over-called or under-called. In an example, the probability of a base being over-called is calculated as a phred scaled log-likelihood ratio between two scores: the highest scored base-space sequence, S, and the score of the sequence containing a one base over-call in positions j, Sⱼ+. Similarly, in an embodiment, the probability of an under-call in position j, S_{J-}. In an example, scores can be calculated by fitting the raw-signal to hypothetical sequence.

$${P}_{j +} = - 10 * log ( 1 - ( \frac{{S}_{j +}}{S} ) )$$

In an example, the intensity value Iⱼ corresponding to a base call at position j of the predicted sequence can be calculated as:

$${I}_{j} = {B}_{j} * 100 + sign ( {S}_{j +} - {S}_{j -} ) \times M \times max ( {P}_{j +} , {P}_{j -} )$$

where Bⱼ is equal to a predicted homopolymer length at j and M is a scaling factor that guarantees that max (Pⱼ₊,P,ⱼ₋) <50. The scaling can convert the probability into a range similar to an intensity value. The intensity corresponding to a no-call can be 0.

In another example, the intensity value Iⱼ corresponding to a base call at position j of the predicted sequence can be calculated as:

$${I}_{j} = A + sign ( {S}_{j +} - {S}_{j -} ) \times M \times max ( {P}_{j +} , {P}_{j -} )$$

where A is a constant that defined granularity and M is a scaling factor that guarantees that max(Pⱼ₊,P,ⱼ₋) <500. In an exemplary embodiment, A can be 500. The scaling can convert the probability into a format standardize by the BAM specification. The intensity corresponding to a no-call can be 0.

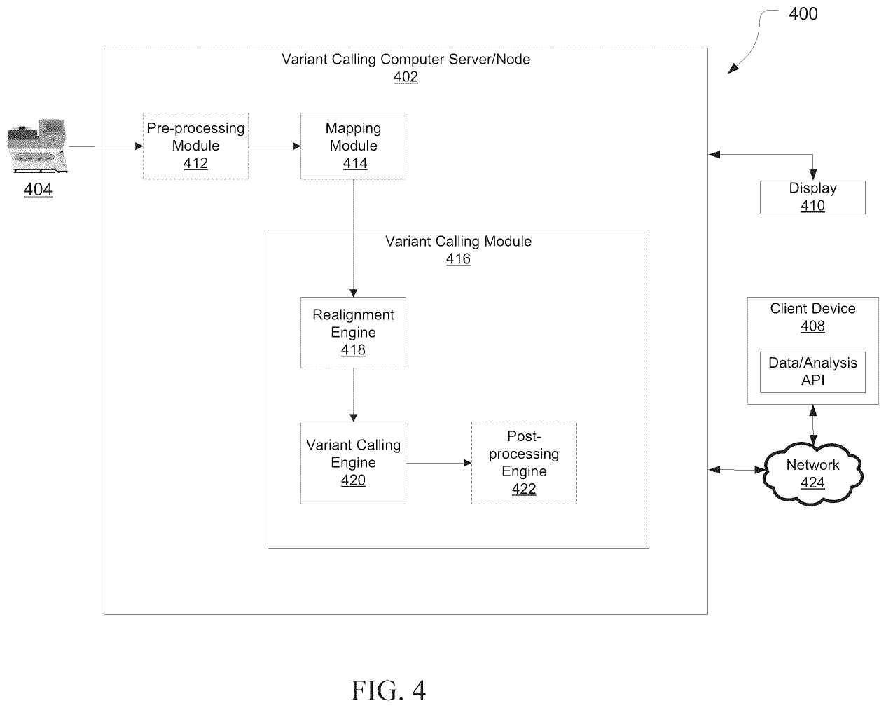

FIG. 4 is a schematic diagram of a system for identifying variants, in accordance with various embodiments.

As depicted herein, variant analysis system 400 can include a nucleic acid sequence analysis device 404 (e.g., nucleic acid sequencer, real-time/digital/quantitative PCR instrument, microarray scanner, etc.), an analytics computing server/node/device 402, and a display 410 and/or a client device terminal 408.

In various embodiments, the analytics computing sever/node/device 402 can be communicatively connected to the nucleic acid sequence analysis device 404, and client device terminal 408 via a network connection 424 that can be either a “hardwired” physical network connection (e.g., Internet, LAN, WAN, VPN, etc.) or a wireless network connection (e.g., Wi-Fi, WLAN, etc.).

In various embodiments, the analytics computing device/server/node 402 can be a workstation, mainframe computer, distributed computing node (part of a “cloud computing” or distributed networking system), personal computer, mobile device, etc. In various embodiments, the nucleic acid sequence analysis device 404 can be a nucleic acid sequencer, real-time/digital/quantitative PCR instrument, microarray scanner, etc. It should be understood, however, that the nucleic acid sequence analysis device 404 can essentially be any type of instrument that can generate nucleic acid sequence data from samples obtained from an individual.

The analytics computing server/node/device 402 can be configured to host an optional pre-processing module 412, a mapping module 414, and a variant calling module 416.

Pre-processing module 412 can be configured to receive from the nucleic acid sequence analysis device 404 and perform processing steps, such as conversion from flow space to base space, determining call quality values, preparing the read data for use by the mapping module 414, and the like.

The mapping module 414 can be configured to align (i.e., map) a nucleic acid sequence read to a reference sequence. Generally, the length of the sequence read is substantially less than the length of the reference sequence. In reference sequence mapping/alignment, sequence reads are assembled against an existing backbone sequence (e.g., reference sequence, etc.) to build a sequence that is similar but not necessarily identical to the backbone sequence. Once a backbone sequence is found for an organism, comparative sequencing or re-sequencing can be used to characterize the genetic diversity within the organism's species or between closely related species. In various embodiments, the reference sequence can be a whole/partial genome, whole/partial exome, etc.

In various embodiments, the sequence read and reference sequence can be represented as a sequence of nucleotide base symbols in base space. In various embodiments, the sequence read and reference sequence can be represented as one or more colors in color space. In various embodiments, the sequence read and reference sequence can be represented as nucleotide base symbols with signal or numerical quantitation components in flow space.

In various embodiments, the alignment of the sequence fragment and reference sequence can include a limited number of mismatches between the bases that comprise the sequence fragment and the bases that comprise the reference sequence. Generally, the sequence fragment can be aligned to a portion of the reference sequence in order to minimize the number of mismatches between the sequence fragment and the reference sequence.

The variant calling module 416 can include a realignment engine 418, a variant calling engine 420, and an optional post processing engine 422. In various embodiments, variant calling module 416 can be in communications with the mapping module 414. That is, the variant calling module 416 can request and receive data and information (through, e.g., data streams, data files, text files, etc.) from mapping module 414. In various embodiments, the variant calling module 416 can be configured to communicate variants called for a sample genome as a *.vcf, *.gff, or *.hdf data file. It should be understood, however, that the called variants can be communicated using any file format as long as the called variant information can be parsed and/or extracted for later processing/analysis.

The realignment engine 418 can be configured to receive mapped reads from the mapping module 414, realign the mapped reads in flow space, and provide the flow space alignments to the variant calling engine 420.

The variant calling engine 420 can be configured to receive flow space information from the realignment engine 418 and fit the distribution of matched-filter residuals to uni-modal and bi-modal models for the homopolymer region. By identifying the closest model, the variant calling engine 420 can detect insertions/deletions in the homopolymers as well as the heterogeneity of the sample.

Post processing engine 422 can be configured to receive the variants identified by the variant calling engine 420 and perform additional processing steps, such as conversion from flow space to base space, filtering adjacent variants, and formatting the variant data for display on display 410 or use by client device 408. Examples of filters that the post-processing engine 422 may apply include a minimum score threshold, a minimum number of reads including the variant, a minimum frequency of reads including the variant, a minimum mapping quality, a strand probability, and region filtering.

Client device 408 can be a thin client or thick client computing device. In various embodiments, client terminal 408 can have a web browser (e.g., INTERNET EXPLORER™, FIREFOX™, SAFARI™, etc) that can be used to communicate information to and/or control the operation of the pre-processing module 412, mapping module 414, realignment engine 418, variant calling engine 420, and post processing engine 422 using a browser to control their function. For example, the client terminal 408 can be used to configure the operating parameters (e.g., match scoring parameters, annotations parameters, filtering parameters, data security and retention parameters, etc.) of the various modules, depending on the requirements of the particular application. Similarly, client terminal 408 can also be configure to display the results of the analysis performed by the variant calling module 416 and the nucleic acid sequencer 404.

It should be understood that the various data stores disclosed as part of system 400 can represent hardware-based storage devices (e.g., hard drive, flash memory, RAM, ROM, network attached storage, etc.) or instantiations of a database stored on a standalone or networked computing device(s).

It should also be appreciated that the various data stores and modules/engines shown as being part of the system 400 can be combined or collapsed into a single module/engine/data store, depending on the requirements of the particular application or system architecture. Moreover, in various embodiments, the system 400 can comprise additional modules, engines, components or data stores as needed by the particular application or system architecture.

In various embodiments, the system 400 can be configured to process the nucleic acid reads in color space. In various embodiments, system 400 can be configured to process the nucleic acid reads in base space. In various embodiments, system 400 can be configured to process the nucleic acid sequence reads in flow space. It should be understood, however, that the system 400 disclosed herein can process or analyze nucleic acid sequence data in any schema or format as long as the schema or format can convey the base identity and position of the nucleic acid sequence.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Further, in describing various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments.

The embodiments described herein, can be practiced with other computer system configurations including hand-held devices, microprocessor systems, microprocessor-based or programmable consumer electronics, minicomputers, mainframe computers and the like. The embodiments can also be practiced in distributing computing environments where tasks are performed by remote processing devices that are linked through a network.

It should also be understood that the embodiments described herein can employ various computer-implemented operations involving data stored in computer systems. These operations are those requiring physical manipulation of physical quantities. Usually, though not necessarily, these quantities take the form of electrical or magnetic signals capable of being stored, transferred, combined, compared, and otherwise manipulated. Further, the manipulations performed are often referred to in terms, such as producing, identifying, determining, or comparing.

Any of the operations that form part of the embodiments described herein are useful machine operations. The embodiments, described herein, also relate to a device or an apparatus for performing these operations. The systems and methods described herein can be specially constructed for the required purposes or it may be a general purpose computer selectively activated or configured by a computer program stored in the computer. In particular, various general purpose machines may be used with computer programs written in accordance with the teachings herein, or it may be more convenient to construct a more specialized apparatus to perform the required operations.

Certain embodiments can also be embodied as computer readable code on a computer readable medium. The computer readable medium is any data storage device that can store data, which can thereafter be read by a computer system. Examples of the computer readable medium include hard drives, network attached storage (NAS), read-only memory, random-access memory, CD-ROMs, CD-Rs, CD-RWs, magnetic tapes, and other optical and non-optical data storage devices. The computer readable medium can also be distributed over a network coupled computer systems so that the computer readable code is stored and executed in a distributed fashion.

1.-24. (canceled)

25. A system for identifying variants in nucleic acid sequence reads, comprising:
a processor configured to:
map a plurality of reads to a reference genome, wherein the plurality of reads for a sample are provided by a base caller;
identify a potential variant position in a homopolymer region of the mapped reads; calculate a plurality of base calling residuals corresponding to the mapped reads that span the homopolymer region using measured values and predicted signal values, wherein the predicted signal values are based on a phasing model for a read and the measured values are based on normalized measurements of sequencing data from a nucleic acid sequencing device;
determine a distribution of the base calling residuals; and detect presence of an insertion/deletion in the homopolymer region and heterozygosity in the sample based on the distribution of the base calling.

26. The system of claim 25, wherein the processor is further configured to fit the base calling residuals distribution to a uni-modal model and a bi-modal model to determine a best-fit model.

27. The system of claim 26, wherein the uni-modal model is a uni-modal Gaussian model and the bi-modal model is a bi-modal Gaussian model.

28. The system of claim 27, wherein the uni-modal Gaussian model includes one Gaussian distribution and the bi-modal Gaussian model includes first and second Gaussian distributions.

29. The system of claim 28, wherein the processor is further configured to, when the best-fit model is the bi-modal Gaussian distribution, identify the sample as heterozygous.

30. The system of claim 29, wherein the processor is further configured to, when the best-fit model is the bi-modal Gaussian distribution, determine a first homopolymer length and a second homopolymer length based on centers of the first and second Gaussian distribution.

31. The system of claim 28, wherein the processor is further configured to, when the best-fit model is the uni-modal Gaussian distribution:
identify the sample as not heterozygous;
determine the homopolymer length a based on a center of the Gaussian distribution; and
output the homopolymer length.

32. The system of claim 25, wherein the processor is configured to calculate the base calling residuals by calculating a matched-filter residual based on normalized measurements y and predicted signals x_{A} and x_{B}, wherein the predicted signal x_{A} is generated using the phasing model applied to a first modified read sequence including a reference homopolymer and the predicted signal x_{B} is generated using the phasing model applied to a second modified read sequence including a second homopolymer that is one base longer than the reference homopolymer.

33. A method for determining a presence or absence of an insertion/deletion variant in a reference homopolymer, comprising:
obtaining sequencing data by a processor, the sequencing data relating to a plurality of template polynucleotide strands disposed in a sample processing unit of a nucleic acid sequencing device, the template polynucleotide strands having been exposed to a series of flows of nucleotide species;
generating one or more preliminary sequences of called bases by performing a preliminary base calling for at least some of the plurality of template polynucleotide strands using the sequencing data;
identifying one or more candidate variant sequences in the one or more preliminary sequences of called bases by mapping the one or more preliminary sequences of called bases against a reference genome; and
calling one or more variants using a distribution of base calling residuals based on measured and model-predicted signal values of the sequencing data, including retrieving, for at least one of the one or more candidate variant sequences, a called sequence c and corresponding normalized measurements y from the sequencing data covering a reference homopolymer h_{A}.

34. The method of claim 33, comprising locating a called homopolymer h_{c} within the called sequence c that aligned to the reference homopolymer h_{A} based on the mapping.

35. The method of claim 34, comprising creating modified read sequences r_{A} and r_{B}, by substituting the called homopolymer h_{c} with the reference homopolymer h_{A} and a homopolymer h_{B}, respectively, where the homopolymer h_{B} is one base longer than the reference homopolymer h_{A}.

36. The method of claim 35, comprising generating a model-predicted signal x_{A} for the modified read sequence r_{A} using one or more phasing parameters and a pre-determined ordering of nucleotide species flows.

37. The method of claim 36, comprising generating a model-predicted signal x_{B} for the modified read sequence r_{B} using one or more phasing parameters and a pre-determined ordering of nucleotide species flows.

38. The method of claim 37, comprising calculating a plurality of base calling residuals based on the normalized measurements y and the model-predicted signals x_{A} and x_{B}.

39. The method of claim 33, wherein obtaining sequencing data further comprises measuring a value representative of a number of incorporation events for at least one of the flows.

40. The method of claim 39, wherein the incorporation events occur when a nucleotide is added to an extending complementary strand.

41. The method of claim 39, wherein measuring includes quantifying an intensity of photons produced in response to the incorporation event.

42. The method of claim 39, wherein measuring includes quantifying a change in an electrical property of a field effect transistor in response to a change in ion concentration due to the incorporation event.

43. A system, including:
a machine-readable memory; and
a processor configured to execute machine-readable instructions, which, when executed by the processor, cause the system to perform a method for determining a presence or absence of an insertion/deletion variant in a reference homopolymer comprising:
receiving at the processor sequencing data relating to a plurality of template polynucleotide strands disposed in a sample processing unit of a nucleic acid sequencing device, the template polynucleotide strands having been exposed to a series of flows of nucleotide species;
generating one or more preliminary sequences of called bases by performing a preliminary base calling for at least some of the plurality of template polynucleotide strands using the sequencing data;
identifying one or more candidate variant sequences in the one or more preliminary sequences of called bases by mapping the one or more preliminary sequences of called bases against a reference genome; and
calling one or more variants using a distribution of base calling residuals based on measured and model-predicted signal values of the sequencing data, including retrieving, for at least one of the one or more candidate variant sequences, a called sequence c and corresponding normalized measurements y from the sequencing data covering a reference homopolymer h_{A}.

44. The system of claim 43, wherein the method further comprises:
locating a called homopolymer h_{c} within the called sequence c that aligned to the reference homopolymer h_{A} based on the mapping;
creating modified read sequences r_{A} and r_{B}, by substituting the called homopolymer h_{c} with the reference homopolymer h_{A} and a homopolymer h_{B}, respectively, where the homopolymer h_{B} is one base longer than the reference homopolymer h_{A};
generating a model-predicted signal x_{A} for the modified read sequence r_{A} using one or more phasing parameters and a pre-determined ordering of nucleotide species flows;
generating a model-predicted signal x_{B} for the modified read sequence r_{B} using one or more phasing parameters and a pre-determined ordering of nucleotide species flows; and calculating a plurality of base calling residuals based on the normalized measurements y and the model-predicted signals x_{A} and x_{B}.
